(19) United States
(12) Patent Application Publication
(10) Pub. No.: US 2010/0216177 A1
(43) Pub. Date: Aug. 26, 2010

(54) METHOD AND KIT FOR UNIVERSAL VERIFICATION OF ENZYME ACTIVITY AND PROTEIN DIGESTION

(75) Inventors: Richard Idem Somiari, Johnstown, PA (US); Stella Bassey Somiari, Johnstown, PA (US); Stephen Russell, Johnstown, PA (US)

Correspondence Address: COHEN & GRIGSBY, P.C., 625 LIBERTY AVENUE, PITTSBURGH, PA, 15222-3152, US

(73) Assignee: ITSI Biosciences, Johnstown, PA (US)

(21) Appl. No.: 12/389,563

(22) Filed: Feb. 20, 2009

(51) Int. Cl. C12Q 1/37

(52) U.S. Cl. 435/23

(57) ABSTRACT

A method of assessing enzyme function or protein stability is disclosed. A sample having a protein therein is contacted with an enzyme that is suspected of being capable of catalyzing digestion of the protein in order to form a mixture. First and second aliquots are removed from the mixture at first and second times, respectively, and protein digestion is evaluated using a detection reagent to calculate a change in absorbance between first and second aliquots. The change in absorbance provides an index of enzyme activity, enzyme capability, and protein stability.

### BACKGROUND

Protein identification and characterization is one of the most important steps in proteomics, including identification and characterization by mass spectrometry. Proteins are typically digested into peptides with enzymes, e.g., trypsin, prior to the mass spectrometry step. However, not all proteins are digested by all enzymes. As a result, some mass spectrometry experiments fail because the target protein was not digested.

### SUMMARY

In an embodiment, a method of assessing at least one of enzyme function and protein stability is disclosed, the method comprising the steps of:
contacting a sample comprised of a protein with an enzyme to form a mixture, wherein the enzyme is suspected of being capable of catalyzing digestion of the protein into a plurality of peptides when the enzyme is active;
inactivating the enzyme in first and second aliquots removed from the mixture at first and second times, respectively; and
evaluating the protein digestion, the evaluating comprising: (i) contacting each aliquot with a detection reagent that is capable of binding to a protein molecule and initiating a color change when bound to the proteins; (ii) measuring an absorbance of each aliquot; and (iii) calculating a change in absorbance between the first and second aliquots, the change being used as an index of at least one of enzyme activity, enzyme capability, and protein stability.

In another embodiment, the method of assessing at least one of enzyme function and protein stability comprises the steps of:
forming a mixture by contacting a sample comprised of a protein with an enzyme, wherein the enzyme is suspected of being capable of catalyzing digestion of the protein into a plurality of peptides when the enzyme is active;
removing first and second aliquots from the mixture at first and second times, respectively, and substantially immediately inactivating the enzyme in each of the aliquots; and
evaluating the protein digestion in each of the aliquots, said evaluating comprising: (i) adding to each aliquot a detection reagent that is capable of binding to protein; (ii) measuring an absorbance of each aliquot; and (iii) calculating a change in absorbance between the first and second aliquots, the change being used as an index of at least one of enzyme activity, enzyme capability, and protein stability.

In another embodiment, the method of assessing at least one of enzyme function and protein stability comprises the steps of:
confirming that the enzyme is active, comprising: (i) contacting a standard comprised of at least one known protein with the enzyme to form a first mixture, wherein the enzyme is known to catalyze digestion of the known protein into a plurality of peptides when the enzyme is active; (ii) removing a first aliquot from the first mixture at a first time and substantially immediately inactivating the enzyme in the first aliquot; (iii) removing a second aliquot from the first mixture at a second time and substantially immediately inactivating the enzyme in the second aliquot; (iv) contacting each of the first and second aliquots with a detection reagent that is capable of binding to protein; (v) reading an absorbance of each of the first and second aliquots following the step of contacting with the detection reagent; and (vi) calculating a change in absorbance between the first and second aliquots, wherein the change is an index of enzyme activity;
evaluating protein digestion, comprising: (i) contacting a biological sample comprising a protein with the enzyme to form a second mixture; (ii) removing a third aliquot from the second mixture at the first time and substantially immediately inactivating the enzyme in the third aliquot; (iii) removing a fourth aliquot from the second mixture at the second time and substantially immediately inactivating the enzyme in the fourth aliquot; (iv) contacting each of the third and fourth aliquots with the detection reagent; (v) reading an absorbance of each of the third and fourth aliquots following the step of contacting with the detection reagent; and (vi) calculating a change in absorbance between the third and fourth aliquots, wherein the change is an index of protein digestion; and
comparing the change in absorbance between the third and fourth aliquots with the change in absorbance between the first and second aliquots.

In another embodiment, a kit for assessing activity of at least one of enzyme function and protein stability is disclosed, the kit comprising:
a standard comprised of at least one known protein that is capable of being digested into a plurality of peptides;
an enzyme inactivating reagent that inactivates the enzyme; and
a detection reagent that binds to the undigested protein. Optionally, the kit further comprises an enzyme. In an example, the enzyme is trypsin or chymotrypsin.

Other objects, features, aspects and advantages of the present invention will become better understood or apparent from the following detailed description, figures, and appended claims of the invention.

### BRIEF DESCRIPTION OF THE FIGURES

FIG. 1 is a flowchart illustrating an embodiment of a method for verifying enzyme activity and protein digestion.

FIGS. 2A and 2B show schematics of embodiments of the method for verifying enzyme activity and protein digestion.

FIG. 3 shows an example of a standard curve used to verify enzyme activity and protein digestion.

FIG. 4 shows an example of a data set generated using an embodiment of the method for verifying enzyme activity and protein digestion. Panel A shows an example of a graph showing absorbance of aliquots of the reaction mixture incubated for various time intervals. Panel B shows an example of an electrophoretic image of the aliquots at the same time intervals.

### DETAILED DESCRIPTION

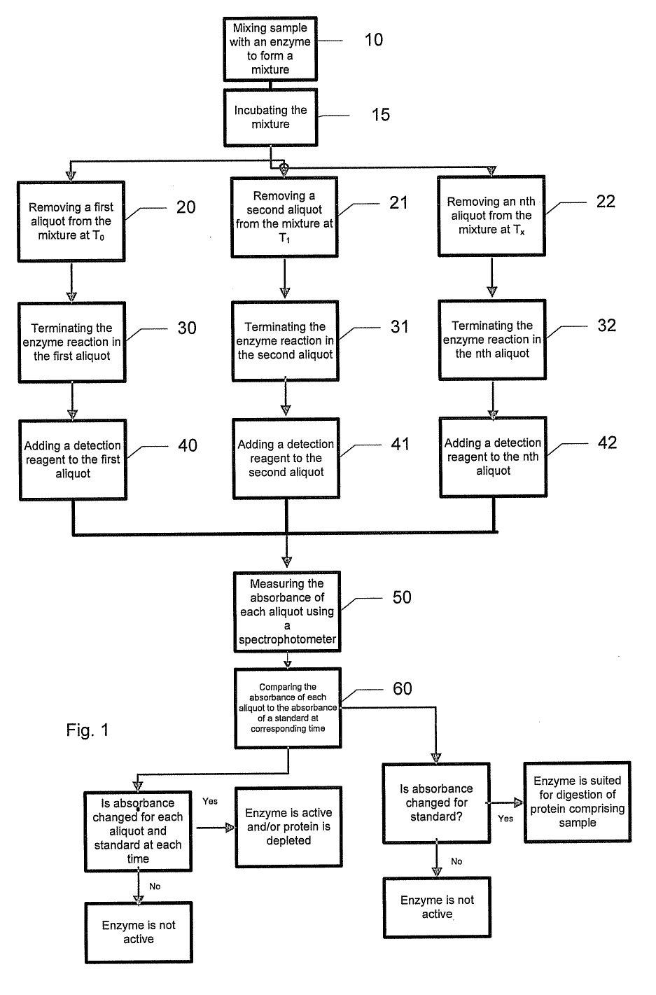

A kit for assessing enzyme activity or protein digestion in known and unknown samples and methods of verifying enzyme activity or protein digestion are disclosed. The method eliminates the need for gel electrophoresis to assess enzyme activity or protein digestion and instead in various embodiments uses a change in absorbance following the addition of a color-generating reagent to aliquots of a reaction mixture comprised of the enzyme and its substrate, the sample, at at least two time points as an index of enzyme activity and protein digestion. See FIG. 1. In an example, the kit and method are used to determine the percentage (%) of protein digested in a sample by calculating the change in absorbance between samples incubated at different time points. In an embodiment, the percentage of protein digested is calculated from the change in absorbance using the equation provided below.

% PD=ΔA_{585 nm}×100 [Equation 1:]

Where ΔA_{585 nm}=[AT₀−A_{TX}/AT₀]100

PD=protein digested

ΔA=change in absorbance at 585 nm

T₀=Time zero

T_{X}=Time at time greater than T₀

### EXAMPLE

If AT0=0.295 and AT 6 h=0.018, then

% PD=[0.295−0.018/0.295]×100=93.9%

A change in absorbance between two aliquots of the enzyme-substrate mixture incubated for different periods of time (T₀ and Tₓ) reflects a change in total protein concentration that indicates that the enzyme is active and capable of catalyzing digestion of the proteins in the sample into peptides and that the proteins comprising the sample were stable and had not broken down prior to contact with the enzyme. In an example, the disclosed kit and methods provide substantially real-time confirmation that the enzyme is functional and able to catalyze degradation of the proteins comprising the sample, or that a sample is suitable for downstream analysis, such as mass spectrometry studies. In an example, the kit and methods provide simultaneous verification of enzyme activity and protein digestion.

The sample is either a pure protein standard or a biological sample. In another example, the sample is a biological sample comprised of at least one protein isolated from any specimen, including for example, blood, tissue, cell lines, plants, insects, bacteria, viruses, and the like. In various embodiments, the biological sample is stored under conditions such as lyophilized or frozen at about −80° C. that substantially prevent protein degradation. In examples, the biological sample is lyophilized or frozen at about −80° C.

The kit comprises a protein standard that is comprised of at least one known purified protein. Known purified proteins are derived, for example, from at least one of membrane proteins, cytoplasmic proteins, nuclear proteins, secreted proteins, ribosomal proteins, and skeletal proteins. Examples of purified proteins include but are not limited to bovine serum albumin, cadherin, glyceraldehydes-3-phosphate dehydrogenase, lamin, and casein. In an example, the pure protein standard comprises a single protein and is used to estimate the activity of the enzyme towards at least one specific protein class. In another example, the standard comprises about three to six known purified proteins mixed together at substantially equal concentrations. The standard comprising the mixture of proteins is substantially representative of the complexity of a biological sample and therefore provides a more accurate single-step estimatation of the enzyme activity in the presence of a mixture of proteins than does a standard comprising a single purified protein. In an example, the standard is provided in the kit in lyophilized form. Optionally, the kit further comprises a buffer for reconstituting the lyophilized standard wherein, the buffer is a composition that supports activity of the enzyme, such as for example 8 M urea with 50 mM Tris (pH=8.5). In another example, the standard is provided in solution in a frozen form.

Optionally, the kit further comprises the enzyme. Alternatively, the enzyme is provided separately from the kit. The enzyme may be any enzyme that is capable of catalyzing the hydrolysis of proteins comprising the sample to form smaller polypeptide units following a contacting period. In an example, the enzyme is trypsin. In another example, the enzyme is chymotrypsin. In an example, the enzyme is a sequencing grade enzyme modified to limit autolysis.

The kit further comprises an enzyme inactivating reagent that inactivates the enzyme and thereby terminates the enzyme reaction following contact of the sample and the enzyme. In an example, the enzyme inactivating reagent comprises about 85% phosphoric acid.

As shown in FIG. 1, the kit further comprises a detection reagent that is capable of binding to the at least one protein comprising the standard or biological sample. The detection reagent is comprised of any dye that binds to the at least one protein. When the detection reagent binds to the at least one protein, a color change occurs in the aliquot and the absorbance of the aliquot at a specified wavelength, such as 585 nm, reflects an overall amount of residual protein present in the aliquot. Therefore, as described in greater detail below, the difference in absorbance in aliquots incubated for first and second times is used as an index of protein digestion or enzyme activity. In an example, the detection reagent is a variant of the standard Bradford protein assay reagent and is comprised of about 250 mg Coomassie® Brilliant Blue 250, about 100 ml of about 85% phosphoric acid, about 50 ml methanol, and about 850 ml distilled water. In another example, the detection reagent binds to a specific class of proteins. Optionally, the detection reagent is fluorescent.

In an embodiment, a method of assessing at least one of enzyme function and protein digestion in a sample is disclosed. A flowchart illustrating an embodiment of the method is shown in FIG. 1. The method is used to confirm that the enzyme is active and capable of catalyzing digestion of at least one of the proteins comprising the sample and that the proteins comprising the sample are stable and have not degraded prior to contact with the enzyme.

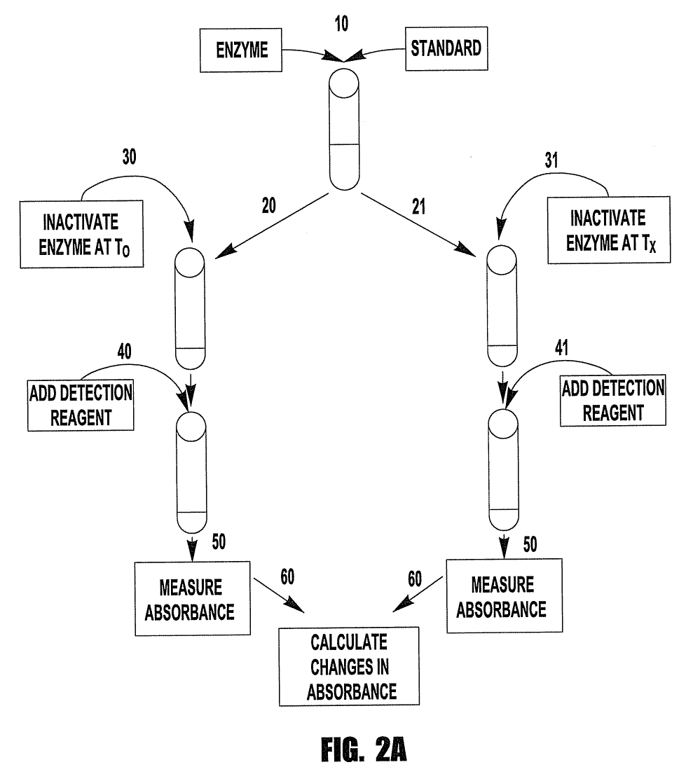

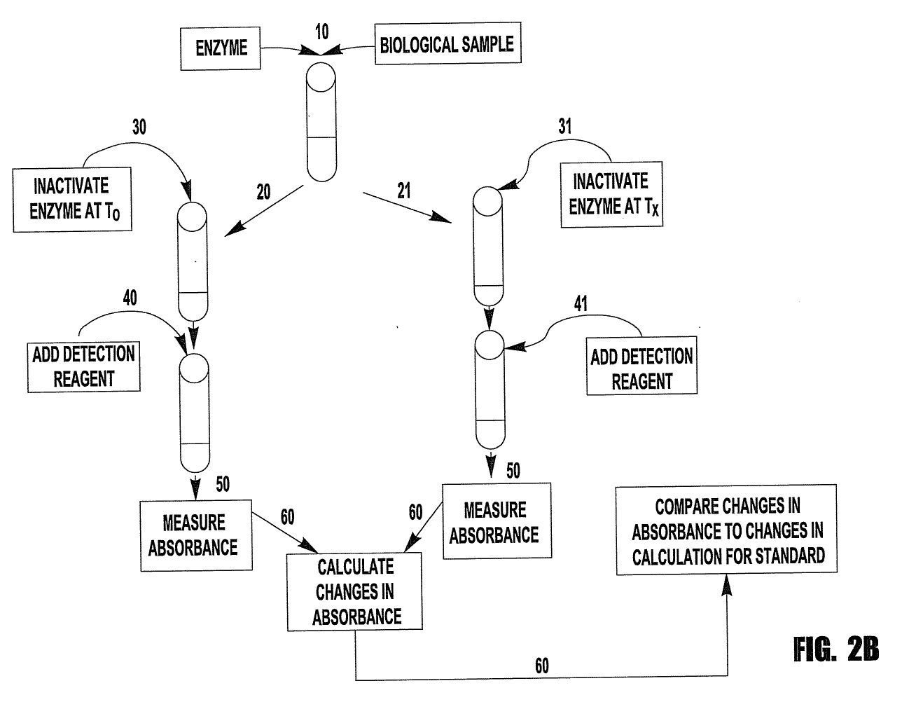

In an optional first step (not shown), the enzyme is reconstituted in the buffer. Next, the method comprises the steps of forming a mixture 10 and incubating the mixture 15, as shown in FIG. 2. In an example using trypsin, the volume of sample used to form the mixture is about 20 μl and contains about 10 μg protein. The volume of enzyme used to form the mixture is about 5 μl to about 10 μl and contains about 1 μg to about 5 μg of enzyme. In examples, the mixture is maintained at about 20° C. to about 25° C. or about 30° C. to about 37° C. However, the volume, incubation time, and incubation temperature vary depending on the enzyme being used.

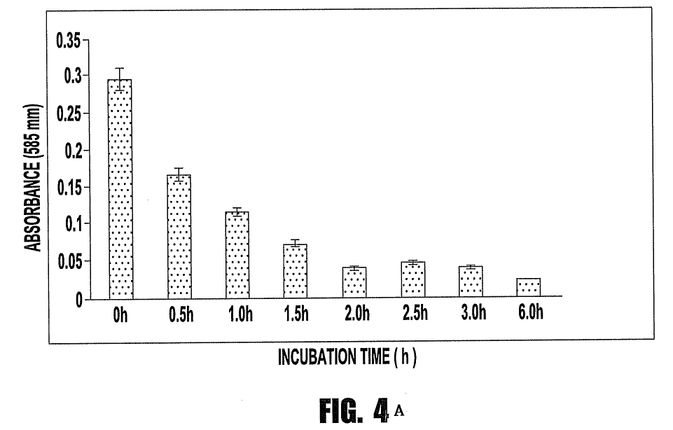

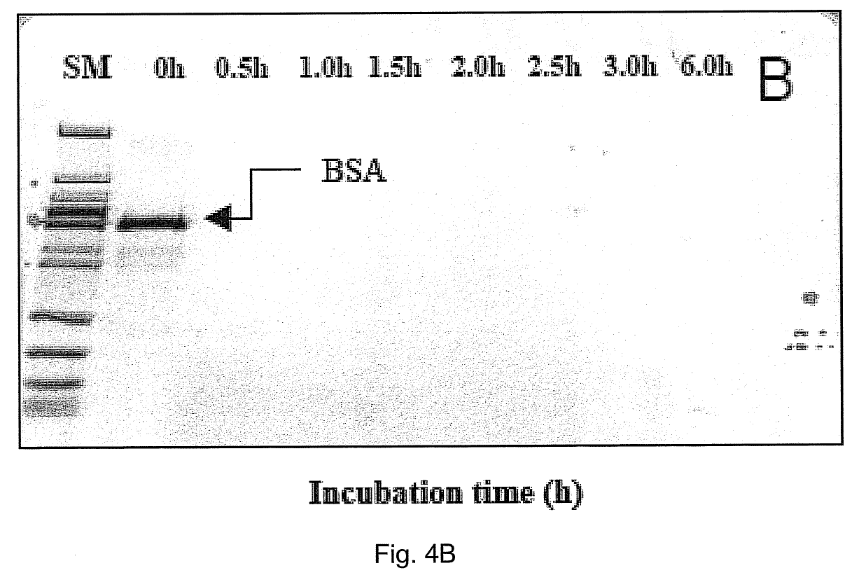

In a next step 20, at a first time, a first aliquot is removed from the mixture. In a next step 30, about 2 μl to about 5 μl of the enzyme inactivating reagent is added to about 5 μl to about 10 μl of the first aliquot to substantially immediately inactivate the enzyme. In an example, the inactivating solution is about 1.7% phosphoric acid or about 0.1M sodium hydroxide. First time can be any time from the time of contact. In an example shown in FIG. 4, first time is zero, defined herein as substantially immediately after the enzyme and sample are mixed together. In other examples, first time is a sufficient period of time of contact between the enzyme and sample for the enzyme to bind and digest at least some of the proteins comprising the sample or standard.

At a second time, a second aliquot is removed from the mixture 21 and the enzyme is substantially immediately inactivated 31 as described above. See FIG. 4. Second time is of a greater duration than first time and is measured from the time of contact. In an example, second time is related to the temperature at which the mixture is maintained, such that if the mixture is maintained at between about 30° C. and about 37° C., then second time is about 3 hours and if the mixture is maintained at between about 20° C. to about 25° C., second time is about 24 hours.

Optionally, as shown in step 22 additional aliquots are removed from the mixture at other times, and the enzyme is substantially immediately inactivated in step 32 as described above. An example of a data set comprised of absorbance measured at additional times is shown in FIG. 4 and is described in greater detail below.

Next, the detection reagent is added to the first aliquot 40, 41, 42. In an example, the volume of detection reagent used is about 200 μl, although other volumes of the detection reagent are contemplated. In an example, the protein detection reagent consists of 50 mg Coomassie Brilliant Blue G250, 100 ml of 85% phosphoric acid, 850 mL of H₂O, and 50 mL methanol. In an example, the detection reagent is in contact with the aliquot for between about 5 and about 30 minutes at about 25° C.

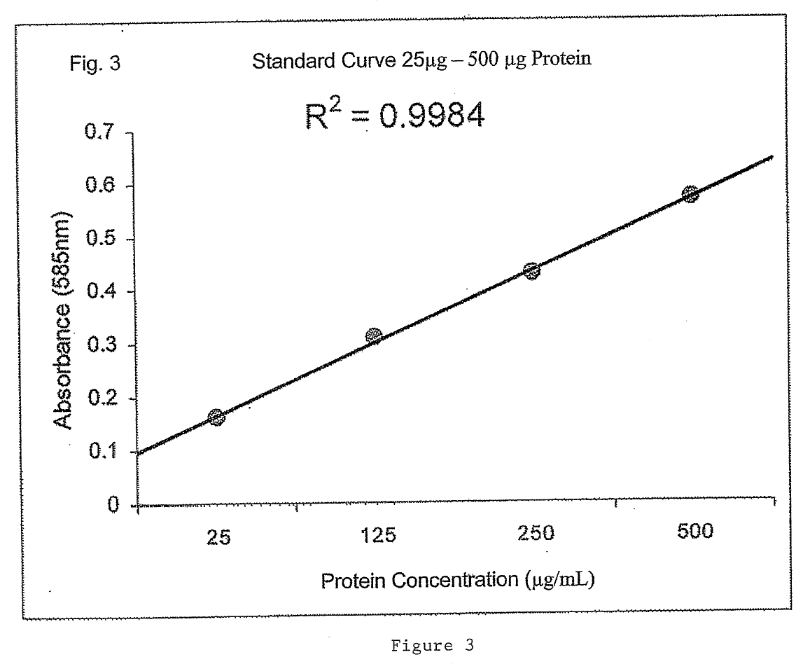

Next, the absorbances of the first and second aliquots are measured using a spectrophotometer 50. In examples, the absorbance is measured at a wavelength of about 585 nm. In other examples, the wavelength can be between about 570 nm to about 595 nm. In an example, the absorbance of each standard is graphed as a function of time and is used to create a standard curve such as the one shown in FIG. 3. In use, the standard curve generated from absorbance readings of aliquots of the standard at first and second time, the standard are used to evaluate protein digestion in a biological sample by comparing absorbance readings of the standard and biological sample at the same first and second times, as shown in step 60.

In a next step 50, the change in absorbance between first and second aliquots is calculated according to Equation 1, set forth above, and the change is used as an index of enzyme activity, enzyme capability, and protein stability 60.

In an embodiment of using the kit shown in FIG. 2, the change in absorbance of aliquots of a standard incubated with the enzyme is compared to the change in absorbance of aliquots of a standard incubated with the enzyme for substantially equal times. As shown in FIG. 2A and as described in greater detail above, a volume of standard and a volume of enzyme are contacted to form a mixture, first and second aliquots of the mixture are removed at times first and second times, T₀ and Tₓ, respectively, and a volume of the detection reagent is added. The absorbance of the resulting solution is measured spectrophotometrically and the change in absorbance between the first and second aliquots is calculated.

As shown in FIG. 2B and as described in greater detail above, a volume of a biological sample and a volume of enzyme are contacted to form a mixture, first and second aliquots of the mixture are removed at times first and second times, T₀ and Tₓ, respectively, and a volume of the detection reagent is added. The absorbance of the resulting solution is measured spectrophotometrically and the change in absorbance between the first and second aliquots is calculated.

The change in absorbance of aliquots of the mixture comprising the biological sample is compared to the change in absorbance of aliquots of the mixture comprising the standard in the following manners:

(1)

| Sample | Change in absorbance | Interpretation |
|---|---|---|
| Standard | if decrease | then enzyme is active |
| Biological sample | if decrease | then enzyme successfully |
| | | digested the sample; continue |
| | | with subsequent analysis |

(2)

| Sample | Change in absorbance | Interpretation |
|---|---|---|
| Standard | if decrease | then enzyme is active |
| Biological sample | no change | the enzyme is active but it is |
| | | not capable of digesting the |
| | | proteins comprising the |
| | | biological sample; |
| | | do not continue with |
| | | subsequent analysis |

(3)

| Sample | Change in absorbance | Interpretation |
|---|---|---|
| Standard | if no change | then enzyme is not active |
| Biological sample | if no change | then enzyme is not active; |
| | | do not continue with |
| | | subsequent analysis |

### EXAMPLE

In an example, the disclosed kit and method according to various embodiments were used to assess the function of trypsin by evaluating the degradation of a sample comprised of bovine serum albumin (BSA). Data are shown in FIG. 4. The trypsin and BSA were mixed together and the mixture was maintained at about 37° C. At first time (about 0 hours) a first aliquot (about 20 μl) was removed from the mixture and the enzyme was inactivated by adding about 10 μl of the enzyme inactivating reagent to the first aliquot. Additionally 20 ml aliquots were removed from the mixture at substantially the following time intervals: 0.5 hour; 1.0 hour; 1.5 hours; 2.0 hours; 2.5 hours; 3.0 hours; and 6.0 hours. Substantially immediately after removing each aliquot, 10 μl of the enzyme inactivating reagent was added to the aliquot to inactivate the enzyme.

Each aliquot was divided into two sub-samples: (1) the solution in one tube was used to measure absorbance; and (2) the solution in the other tube was used for electrophoresis in order to demonstrate that trypsin digestion of BSA leads to a reduction in absorbance using the disclosed method.
(1) About 1 ml of detection reagent was added to one of the sub-samples of each aliquot and the resulting solution was incubated for about 5 minutes. The absorbance of the solution was measured spectrophotometrically at about 585 nm. The spectrophotometric readings of each aliquot at each time period are shown in FIG. 4A.
(2) One dimensional SDS-polyacrylamide gel electrophoresis was performed on corresponding sub-samples of each aliquot. The gel was stained with a fluorescent dye and the gel image was captured with a digital image shown in FIG. 4B.
   As shown in FIG. 4, the BSA protein band in Panel B is mainly visible at time 0, indicating that there has not been any protein digestion at this time. The decrease in absorbance (Panel A) corresponds with the disappearance of the gel band representing BSA (Panel B). These data confirm that digestion of BSA by trypsin leads to a decreased absorbance.

While embodiments of the invention have been shown and described with particularity in connection with the accompanying figures, the invention may be otherwise embodied within the scope of the following claims.

We claim:

1. A method of assessing at least one of enzyme function and protein stability, the method comprising the steps of:
a. forming a mixture by contacting a sample comprised of a protein with an enzyme, wherein the enzyme is suspected of being capable of catalyzing digestion of the protein into a plurality of peptides when the enzyme is active;
b. removing first and second aliquots from the mixture at first and second times, respectively, and substantially immediately inactivating the enzyme in each of the aliquots; and
c. evaluating the protein digestion in each of the aliquots, said evaluating comprising: (i) adding to each aliquot a detection reagent that is capable of binding to at least one of the peptides; (ii) measuring an absorbance of each aliquot; and (iii) calculating a change in absorbance between the first and second aliquots, the change being used as an index of at least one of enzyme activity, enzyme capability, and protein stability.

2. The method as in claim 1 wherein the enzyme is selected from the group consisting of trypsin and chymotrypsin.

3. The method as in claim 1 wherein the enzyme is modified to limit autolysis.

4. The method as in claim 1 wherein at least one of the steps of forming, inactivating, and evaluating is carried out at between about 25° C. and about 37° C.

5. The method as in claim 1 wherein the step of measuring the absorbance is carried out at a wavelength between about 570 nm and about 595 nm.

6. The method as in claim 1 wherein the step of calculating the change in absorbance is preformed according to the following formula:
PD=[(A_{1(T0, λ})−A_{2(Tx, λ)})/A_{1(T0, λ)}]*100,
wherein PD is a percent of the protein digested into the plurality of peptides,
wherein A_{1(T0, λ)} is the absorbance of the first aliquot at the first time at a wavelength, and
wherein A_{2(Tx, λ)} is the absorbance of the second aliquot at the second time at the wavelength.

7. The method as in claim 1 wherein the sample is a standard comprised of at least one known protein.

8. The method as in claim 1 wherein the change in absorbance is used to confirm that the enzyme is active.

9. The method as in claim 1 wherein the sample is a biological sample comprised of at least one unknown protein.

10. The method as in claim 1 wherein the change in absorbance is used to confirm that the enzyme is capable of catalyzing digestion of the protein.

11. The method as in claim 1 wherein the change in absorbance is used to confirm that the protein comprising the sample is stable.

12. The method as in claim 1 wherein the change is a decrease.

13. A kit for assessing activity at least one of enzyme function and protein stability, the kit comprising:
a. a standard comprised of at least one known protein that is capable of being digested into a plurality of peptides;
b. a standard buffer that is used to resuspend the standard into a solution;
c. an enzyme inactivating reagent that inactivates the enzyme; and
d. a detection reagent that binds to the protein and allows the measurement of absorbance.

14. The kit as in claim 13 wherein the at least one known protein is selected from the group consisting of membrane proteins, cytoplasmic proteins, nuclear proteins, universal proteins, secreted proteins, ribosomal proteins, and skeletal proteins and used as standard.

15. The kit as in claim 13 further comprising a buffer that is used to reconstitute the standard.

16. The kit as in claim 13 further comprising the enzyme.

17. The kit as in claim 16 wherein the enzyme is selected from the group consisting of trypsin or chymotrypsin.

18. The kit as in claim 16 wherein said enzyme is modified to limit autolysis.

19. The kit as in claim 13 wherein the standard is comprised of a plurality of proteins selected from the group consisting of bovine serum albumin, cadherin, glyceraldehydes-3-phosphate dehydrogenase, lamin, and casein.

20. The kit as in claim 13 wherein the standard is at least one of lyophilized or in solution.
